(19) United States
(12) Patent Application Publication
(10) Pub. No.: US 2015/0056096 A1
(43) Pub. Date: Feb. 26, 2015

(54) Self-Cleaning Beverage Nozzle

(71) Applicant: George Harold Hoover, Carmel, CA (US)

(72) Inventor: George Harold Hoover, Carmel, CA (US)

(21) Appl. No.: 14/462,326

(22) Filed: Aug. 18, 2014

Related U.S. Application Data
(60) Provisional application No. 61/959,447, filed on Aug. 24, 2013.

(51) Int. Cl. B67D 1/07; A61L 2/10; B67D 1/00

(52) U.S. Cl. CPC B67D 1/07; B67D 1/0021; A61L 2/10; B67D 2001/075
USPC 422/24; 222/148

(57) ABSTRACT

The present invention relates to a self-cleaning nozzle. The nozzle is coated with a photocatalytic material and a UV light source is used to activate a chemical reaction that kills microbacteria. The nozzle may be made of a translucent material that allows UV light to enter the nozzle.

### CROSS REFERENCE TO RELATED APPLICATIONS

This application is related to, and claims the benefit of, Provisional Patent Application No. 61/959,447, filed on Aug. 18, 2013, entitled “Photocatalysts: Nozzle And Diffuser Cleaning and Disinfecting,” which is hereby incorporated by reference as if fully set forth herein.

### FIELD OF THE INVENTION

The present invention relates to a self-cleaning nozzle, and in particular to a self-cleaning nozzle using a nano photocatalytic material that includes both antimicrobial and hydrophilic properties. Such coatings provide two important functions. First, hydrophobic surfaces are difficult for microbial and other containments to attach Thereby the normal liquid flow through the nozzle flushes them away. Second, the proposed antimicrobial actions aggressively destroy any remaining microbial matter.

### BACKGROUND OF THE INVENTION

Fountain beverage dispensing nozzles have a history of containing and growing microbial matter, some of which are toxin to humans. A recent study by Reder Godard published in the Journal of Food Microbiology suggested that 48% of soda fountains they tested are contaminated with coliform bacteria. In total, approximately 20% of soda fountains tested had more than the maximum recommended level of bacteria. Additionally, fungus may often be found in nozzles which may grow into large colonies that when dispensed into a beverage often provokes a very negative customer reaction. The combination of water and syrup provides an optimal breeding environment for microbials in the nozzle.

In order to address the problem, the owners/operators of the soda fountain equipment must remove and sanitize the nozzles and diffuser valve assemblies on a daily basis. Nozzle assembly sanitization, which is proscribed by beverage syrup companies, results in not only wasted labor costs but also wasted beverage syrup. Syrup waste per valve is approximately 5 ounces per cleaning or about 14 gallons per year per valve. Several hours of store labor are also required per valve each year. For a retail outlet having 20 dispensing valves, the annual expense associated with cleaning the nozzle may be in the range of approximately $4,000 per year.

The daily nozzle sanitization procedures recommended by The Coca-Cola Company are summarized below.

1. Wash hands.

2. Prepare 2.5 gallons of chlorine based sanitizer in a dedicated bucket.

3. Remove nozzles and diffusers.

4. Clean removed nozzles and diffusers in sanitizing solution with brush.

5. Air dry.

6. Use brush to clean underneath valve body.

7. Wash hands.

8. Reinstall nozzles and diffusers.

9. Flush each valve for TEN seconds (typically 25 oz. of water and 5 oz. of syrup).

Other syrup suppliers have similar recommended procedures for cleaning the nozzle and diffuser valve assemblies on a daily basis.

The current process for cleaning nozzles and diffusers is costly and labor intensive. It is important to maintain nozzles as clean as possible. The flow of soda leaves a residue on the inside of the nozzles. Without regular cleaning, this residue, if contaminated may grow into toxic germ levels, and could potentially make people sick. The build up within the nozzle may attract insects that in addition to being annoyingmay carry undesirable germs themselves. Constant neglect will likely lead to dangerous levels of microbial growth.

### OBJECT OF THE INVENTION

This invention is designed to eliminate the daily requirement of sanitizing nozzles and diffusers. The concept presented in this application is designed to provide its cleaning and long term sanitizing, however, it is also anticipated that it may be used to extend the interval between cleaning or nozzle replacement—thus eliminating syrup and labor waste. The concept of this invention is to coat surfaces inside the nozzle space (nozzle walls, valve surfaces, diffuser, etc.) with a nano photocatalytic surface, that when activated by ultraviolet light (UV light) and water, kills pathogens and VOCs. Ultraviolet (UV) light is electromagnetic radiation with a wavelength shorter than that of visible light but longer than x-rays, that is, in the range between approximately 400 nm and 10 nm, corresponding to photon energies from 3.1 eV to 124 eV. Some antimicrobial coatings anticipated for use with this patent are activated by light in the 400 to 600 nm wave lengths. Another benefit of the nano photocatalytic surface is that it develops a hydrophilic surface that sheds water and other contaminants. This double action acts to maintain the nozzle in a clean state. The invention provides the needed light and water to activate the coatings in the normally dark nozzle area.

### SUMMARY OF THE INVENTION

In accordance with the preferred embodiment of the present invention, a fountain beverage dispense system having a self-cleaning nozzle comprising a nozzle having a wall consisting of an exterior and interior surface; the nozzle forming a chamber within the interior surface; a syrup inlet and diffuser located within the chamber of the nozzle; the interior wall of the chamber, the syrup inlet and the diffuser are treated with a photocatalytic nano-material that generates electrons on a treated surface of the nozzle; the nozzle material acts as a light portal and is generally translucent to admit and diffuse UV light energy into the chamber where the UV light energy “causes” oxygen molecules to react with the electrons on the surface to form a superoxide anion which counteracts and decomposes bacteria within the chamber of the nozzle. In accordance with the present invention, the nozzle cleaning action occurs through a photocatalytic reaction.

According to the present invention, a photocatalytic reaction is initiated in a nozzle assembly treated with the nano photocatalytic material when exposed to UV light. Exposure to UV light causes a photoexcited electron to be promoted from the filled valence band of a semiconductor photocatalyst (SC) to the empty conduction band as the absorbed ultraviolet light energy, hv, equals the band gap of the semiconductor photocatalyst, leaving behind a hole in the valence band. In concert, electron and hole pair (e⁻-h⁺) is generated. The following chain reactions have been widely accepted:

Photoexcitation: TO₂/SC+hv→e⁻+h⁺ (1)

Oxygen ionosorption: (O₂)_{ads}+e⁻→O_{2.}⁻ (2)

Ionization of water: H₂O→OH⁻+H⁺ (3)

Protonation of superoxides: O_{2.}⁻+H⁺→HOO. (4)

The hydroperoxyl radical formed in (4) also has scavenging properties similar to O₂ thus doubly prolonging the lifetime of a photohole:

HOO.+e⁻→HO₂⁻ (5)

HOO⁻+H⁺→H₂O₂ (6)

Both the oxidation and reduction can take place at the surface of the photoexcited semiconductor photocatalyst. Recombination between electron and hole occurs unless oxygen is available to scavenge the electrons to form superoxides (O_{2.}⁻), its protonated form the hydroperoxyl radical (HO₂.) and subsequently H₂O₂. The hydroxyl radicals and singlet oxygen are orders of magnitude more toxic to pathogens than chlorine or ozone.

It is another object of the present invention that the photocatalyst reaction eliminates the pathogens from the nozzle assembly by hydrophilic action. The combined antimicrobial and hydrophilic action are designed to eliminate the need for nightly sanitation. It is noted that if the described action keeps toxic microbial below toxic levels, cleaning will be extended or eliminated.

Yet another advantage of the present invention can be found in the fact that many operators of fountain drink dispensing equipment do not sanitize the nozzles on a regular basis. Thus, utilizing the present invention, valves incorporating the described nozzle assemblies would be maintained in a more sanitary environment.

Finally, operators of fountain drink dispensing equipment occasionally avoid adequately flushing the nozzle with beverage product after the sanitation process which results in a undesirable chlorine taste of the first few beverages dispensed from the system. The present invention does not contribute to an off-taste associated with a chlorinated sanitization system.

In this application, the terms “beverage syrup,” “syrup” and “concentrate” are used to describe the ingredients that are mixed with a diluent to create a fountain beverage product. This invention is not limited to these ingredients, but also covers any ingredients or combination of ingredients which may be mixed in a nozzle assembly. Fountain dispense systems originally used sugar-based “syrups,” and later introduced artificially sweetened “concentrates.” The invention of the present application pertains to any beverage ingredient(s) that may be combined to produce a fountain beverage, as such, the terms “fountain beverage,” “beverage,” or “beverage product” means any product produced from a fountain beverage dispenser. The terms “diluent” or “water” refers to carbonated water, non-carbonated water and/or any other diluent mixed with the beverage ingredients to yield a beverage. The term “translucent” means transparent, clear, sheer or having the ability to admit, transmit, and diffuse UV light energy or photon energy. The term “ultraviolet light” means electromagnetic radiation with a wavelength shorter than that of visible light but longer than x-rays, that is, in the range between approximately 400 nm and 10 nm, corresponding to photon energies from 3.1 eV to 124 eV, or substantially close to that range.

### DESCRIPTION OF THE FIGURES

Attention is now directed to drawings that illustrate the features of the present invention:

FIG. 1 is a representation of a conventional fountain beverage dispensing nozzle assembly mounted on a dispensing valve.

FIG. 2 is an exploded partial view of a conventional fountain beverage dispensing nozzle assembly and valve.

FIG. 3 is a cross-section view of the fountain beverage dispensing nozzle and syrup tube of the current invention.

FIG. 4 is a chemical photocatalytic reaction.

FIG. 5a is an alternative embodiment of the present invention including a dedicated light source.

FIG. 5b is an alternative embodiment of the present invention including a partial light portal.

FIG. 6 is an alternative embodiment of the present invention including a built-in LED light source.

The scope of the present invention is not limited to what is shown in the figures.

### DETAILED DESCRIPTION OF THE PREFERED EMBODIMENT

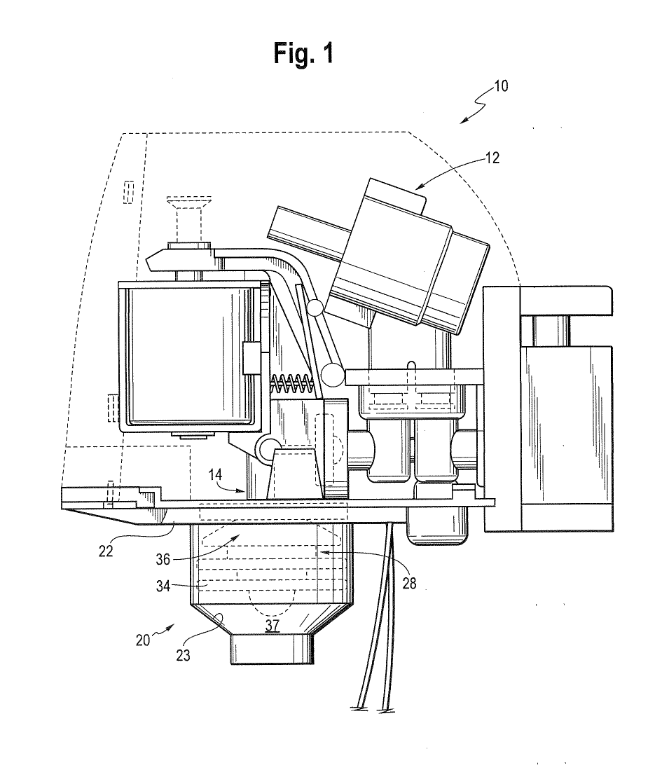

A schematic representation of a conventional fountain beverage dispensing valve 10 is shown in FIG. 1. The nozzle 20 is located at the lower portion of the dispensing valve 10. The nozzle may be made of plastic, glass, metal or other suitable material. Flow control valves 12 regulate the flow of the desired diluent and syrup into the nozzle 20.

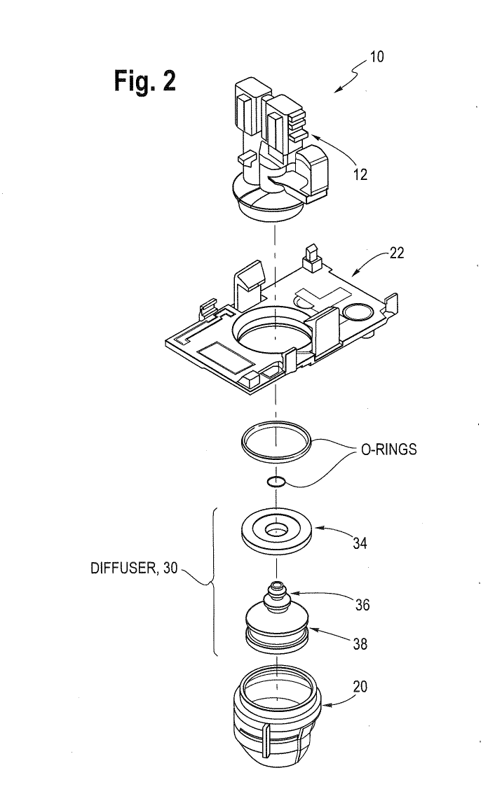

The exploded view of the fountain beverage dispensing valve 10 is shown in FIG. 2. The flow controls 12 regulate the flow of a diluent and syrup into the nozzle 20. The nozzle 20 is typically attached to a base plate 22. Also attached to either the base plate 22 or the nozzle 20 is a diffuser 30. The diffuser may include a diffuser disk 34. A syrup tube 36 with a diffuser plate 38 are also located in the nozzle.

In the typical operation of the beverage dispensing valve 10, a user may select to dispense a desired soft drink product such as Coca-Cola® or Pepsi®. In a typical fountain beverage dispensing system, concentrate beverage syrup from a supply of syrup is delivered by a syrup pump through tubing to a chiller. The beverage syrup, normally contained in a bag-in-box package, is stored in a remote location from the beverage dispensing equipment. The chiller may be a water bath chiller or a cold plate. The syrup is delivered through tubing from the chiller equipment to a valve, or flow control device. The syrup is propelled through the system by a syrup pump. While a single syrup supply is being described, it is well understood that fountain beverage dispensers normally include a plurality of syrup supplies and associated syrup flow circuits.

A water line, which usually connects to a city main, delivers water to an inlet to a carbonator, within which water is carbonated in a manner well understood in the art. Carbonated water exiting the carbonator flows through tubing to and through at least one circuit of the chiller, within which it is desirably chilled to a temperature near 32° F. Upon exiting the chiller, the carbonated water flows to and through a water flow control device to the beverage dispense nozzle. As is understood, the syrup and water flow rate valves 12 operate to meter the flow rates of syrup and water so that a selected ratio of water and syrup is delivered to the dispensing nozzle for exit through an outlet from the nozzle and introduction into a cup positioned beneath the nozzle. Thus, if a Coca-Cola® product is selected by the user, the syrup for a Coca-Cola® product is pumped to the syrup flow control valves 12 and distributed to the nozzle at the desired rate. A typical nozzle and flow control valve arrangement can be found in U.S. Pat No. 5,269,442 entitled “Nozzle For A Beverage Dispensing Valve” which is herein incorporated by reference.

In the prior art dispensing nozzles, a diluent was dispensed through the flow control 12 and would proceed through an outlet 14 into the chamber 28 of the nozzle 20. The syrup was dispensed through the flow control 12, through a syrup tube 36 and dispensed into the nozzle at the outlet 37 of the syrup tube 36. After a beverage product is dispensed through the nozzle, remnants of water and syrup may remain in the nozzle chamber 28 on the interior wall 23, the diffuser 34, the syrup tube 36 and base plate 22.

The combination of water and sugar and/or other organic ingredients create the perfect breeding environment for bacteria. The microbial matter can establish itself on the plastic components if not properly maintained. Without routine sanitation with chlorine or other disinfectants, the nozzle may become increasingly contaminated with undesirable microbial matter.

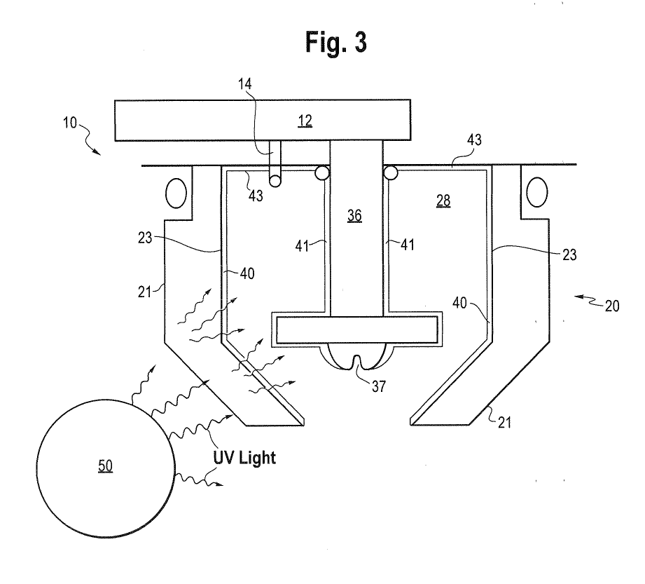

FIG. 3 shows the present invention of a self-cleaning nozzle assembly. In the present invention, the nozzle assembly 10 comprises a nozzle 20, diffuser 30, syrup tube 36; all wetted nozzle assembly surfaces. The nozzle has an exterior surface 21 and an interior surface 23. The interior surface 23 of the nozzle 20 forms a nozzle chamber 28. The syrup tube 36 has an inlet portion that protrudes through the base plate 22. The syrup tube 36 also has an outlet 37 that dispenses the syrup into the nozzle chamber 28. The water inlet 14 also dispenses water into the nozzle chamber 28. The specific portions of syrup and water to be mixed in the nozzle chamber 28 are controlled by flow controls 12.

The interior surface 23 of the nozzle assembly is coated with a nano photocatalytic surface 40. Optionally, the syrup tube 36, the diffuser 30, and the outlet 37 may be treated with a nano photocatalytic surface 41. The base plate 22 may also have a nano photocatalytic surface 43. The material of the nozzle 20 is constructed of translucent plastic material which allows UV light (photon energy) from a light source 50 to pass from the exterior surface 21 of the nozzle 20 through the interior surface 23 of the nozzle 20, such that UV light is present within the chamber 28 and contacts the nano photocatalytic surface 40 on the interior of the nozzle 20. The nozzle 20 may have a light portal (not shown) to permit the UV light to enter the chamber 28. The interior surface 23 of the nozzle may also have a reflective coating to distribute the UV light within the chamber 28. The UV light in the chamber 28 may also contact the nano photocatalytic surface 41 deposited on the syrup tube 36, diffuser 30 and outlet 37 of the syrup tube 36.

Typically, an ambient light emitting UV radiation from an outside light source and water from the dispensed beverage activates the photocatalytic surface 40 and/or 41. The photocatalytic surface could be deposited or adhered to all surfaces of the nozzle assembly in which a diluent and/or syrup contacts. Adequate ambient light 50 enters the interior chamber 28 of the nozzle 20 because the nozzle 20 is constructed of a clear, translucent or transparent material. Water from the beverage composition may be used to activate the photocatalytic reaction. A benefit of using beverage water and ambient light to activate the coating is that antimicrobial and cleaning action occurs each time the valve is used. The need for separate water flushing is eliminated.

One example of a nano photocatalytic coating 40, 41 and 43 material that may be used is made of nano particles of titanium dioxide, silicon oxide or zinc. The photocatalytic material may also be an antimicrobial nano coating. The nano photocatalytic material may consist of a nano layer such as Ag/TiO₂, Au/TiO₂ or Pb/TiO². Titania is another material that has been used as a photocatalyst for generating charge carriers, thereby inducing reactive and oxidative processes. An advantage of using titanium dioxide and similar coatings is that they are activated by fluorescent light frequencies emitting UV light commonly found in retail outlets. Thus, by creating a method to get ambient light into the dark nozzle space, other artificial means are not needed. A known antimicrobial coating that may be used as the photocatalytic surface is sold under the brand name “Oxititan.”

The photocatalytic surface 40 acts as a self-cleaning anti-bacterial anti-fungal surface. It purifies the surrounding materials. Employing a light catalyst or photocatalyst, a light source emits light in the UV spectrum (“UV light”) to activate an oxidation process so any germs, algaes or molds will be transformed to non-harmful molecules. The photocatalyst not only kills bacteria cells, but also decomposes the cell itself. The titanium dioxide photocatalyst is more effective than any other antibacterial agent, because the photocatalytic reaction works even when there are cells covering the surface while the bacteria are actively propagating. The end toxin produced at the death of cell is also expected to be decomposed by a photocatalytic action. Titanium dioxide does not deteriorate and shows a long-term anti-bacterial effect. Generally speaking, disinfections by titanium dioxide are three times better than chlorine, and 1.5 times better than ozone.

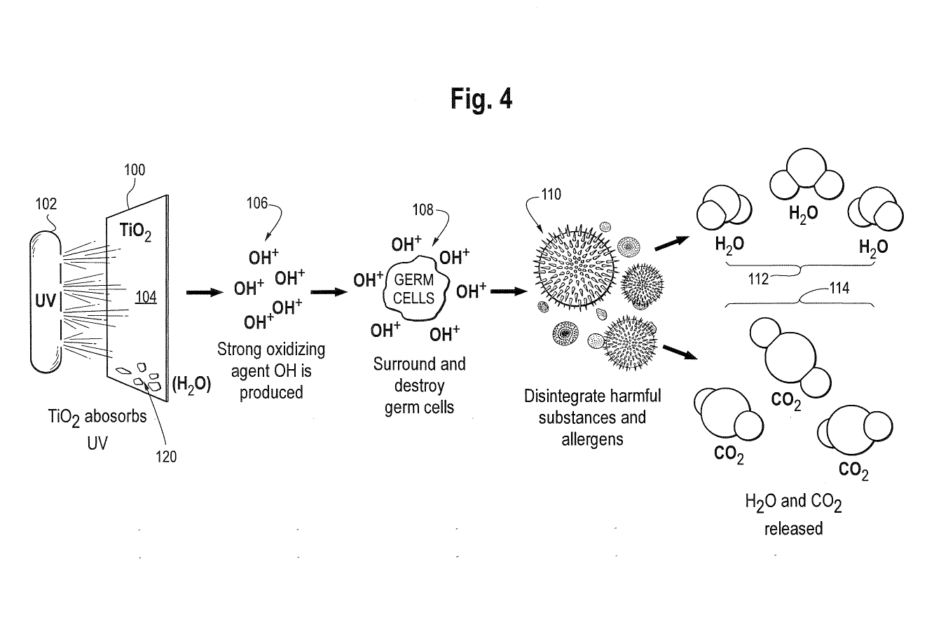

FIG. 4 illustrates the toxic and cleaning action of a typical titanium dioxide based coating 100. A UV, or near UV, light source 102 must be present. When the coated surface 104 interacts with water 120 in the presence of the UV light source (photon energy) 102, a chemical reaction occurs 106 to create two highly reactive substances, hydroxyl radicals (OH⁺) and super oxidized ion. These strong oxidants attract viruses, pathogens and other volatile organic compounds 108. The hydroxyl radicals (OH⁺) and super oxidized ions (shown in 106) act to break down the virus, pathogen or other volatile organic compound into carbon dioxide (CO₂) or water (H₂O) molecules 110. As shown in FIG. 3, the thin active nano photocatalytic surface 40 makes it easy to flush water and other contaminants from the interior surface 23 of the nozzle 20.

In FIG. 4, the nano photocatalytic surface 104 absorbs UV light 102 which creates a chemical reaction in which a strong oxidizing agent OH⁺ is produced 106 from the surface 104. The oxidizing agent OH⁺ 106 acts to destroy germ cells 108 by disintegrating the harmful substances 110. The end result is that the oxidizing agent OH⁺ breaks the virus molecule into water (H₂O) 112 and carbon dioxide (CO₂) 114 molecules which are not harmful to the environment.

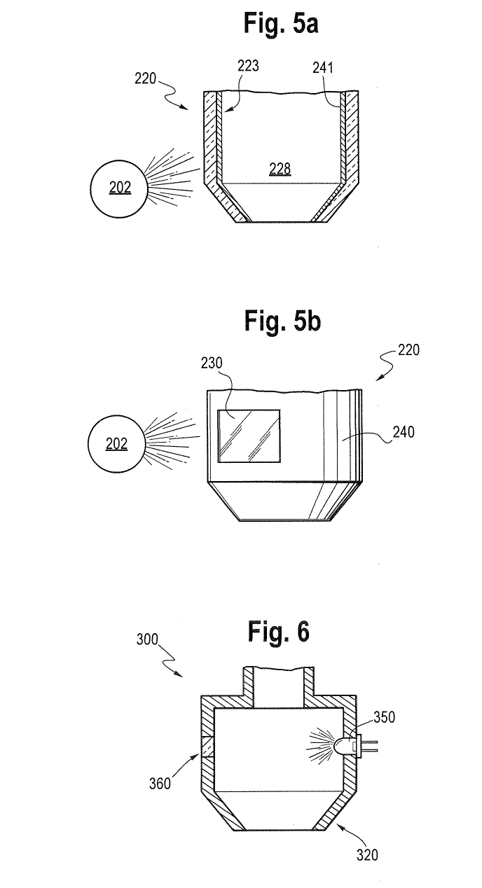

FIGS. 5(a) and 5(b) depict examples of alternative embodiments in which the beverage dispensing system includes a dedicated light source to emit light in the UV spectrum. In the first embodiment of FIG. 5(a), a fluorescent tube light source 202 (or an LED or other UV emitting light source) may be mounted near the nozzle 220. The nozzle 220 may either be made of a translucent plastic material which allows UV light to pass from the exterior surface 221 of the nozzle 220 to the interior surface 223 of the nozzle 220, such that UV light is present within the chamber 228 and contacts the nano photocatalytic surface 241 on the interior of the nozzle 220. Alternatively, only a portion of the nozzle may be made of a translucent plastic material while the remaining portion of the nozzle may be made of an opaque material.

In FIG. 5(b), the nozzle 220 may include a UV light portal made of translucent material 230 to permit UV light to radiate into the nozzle at specified areas and have an opaque nozzle portion 240.

FIG. 6 depicts a further embodiment of the self-sanitizing nozzle assembly 300 which includes a nozzle 320 similar to the nozzles described earlier in this application. The nozzle 320 includes a removable low voltage LED light source 350. The light source 350 may be removably attached to the nozzle 320 by either a screw or compression retention mechanism. Additionally, the embodiment of the nozzle may include an optional window 360 with a lens to indicate that the LED light source is operating properly. If desired, the LED light 350 may be molded into or permanently attached to the nozzle or nozzle assembly.

Given this concept's potential low cost it is anticipated to be used with other nozzle cleaning approaches. For example, an ozone flush may be utilized after hours or at other preset times. This concept could be used with the ozone flush to provide cleaning action throughout the day further enhancing the ozone flush. The anti-pathogenic properties of the proposed photocatalytic coatings are actually more sanitary than ozone.

While embodiments of the invention have been described in detail, various modifications and other embodiments thereof may be devised by one skilled in the art without departing from the spirit and scope of the invention, as defined in the appended claims.

I claim:

1. A self-cleaning nozzle assembly comprising:
a nozzle having an inner surface defining a nozzle chamber wherein at least a portion of said nozzle comprises an ultraviolet light portal;
the nozzle having a top end opening and a bottom drink dispensing opening;
a syrup delivery apparatus that introduces a syrup to said nozzle chamber;
a diluent delivery apparatus that introduces a diluent to said nozzle chamber wherein said diluent is mixed with said syrup;
a nano photocatalytic material adhered to the interior surface of the nozzle assembly that reacts to ultraviolate light to create an oxidizing agent.

2. The apparatus of claim 1, wherein said nano photocatalytic material covers the entire interior surface of the nozzle assembly.

3. The apparatus of claim 1, wherein said ultraviolet light portal is constructed of translucent material.

4. The apparatus of claim 1, wherein said ultraviolet light portal is constructed of clear material.

5. The apparatus of claim 1, wherein said entire nozzle is constructed of translucent material.

6. The apparatus of claim 1, wherein the nano photocatalytic material comprises titanium dioxide.

7. The self-cleaning nozzle apparatus of claim 1, wherein the nano photocatalytic material is selected from the group consisting of Ag/TiO2, Au/TiO₂, or Pb/TiO₂.

8. The apparatus of claim 1, wherein the nano photocatalytic material comprises titania.

9. A method for sanitizing a nozzle, comprising the step of:
using a UV light to radiate a nozzle having a hydrophilic interior surface coated with a photocatalytic layer.

10. The method of claim 9, wherein a photocatalytic layer coating covers the entire interior surface of the nozzle.

11. The method of claim 9, wherein the entire nozzle is constructed of translucent material.

12. The method of claim 9, wherein a portion of the nozzle is constructed of translucent material.

13. The method of claim 9, wherein the UV light radiates the interior chamber formed by the nozzle.

14. A self-cleaning nozzle assembly comprising:
a nozzle having an interior surface defining a chamber, said interior surface coated with a nano photocatalytic material.

15. The self-cleaning nozzle assembly of claim 14 further comprising a UV light portal wherein a UV light radiates into the chamber to initiate a reaction with said photocatalytic material.

16. The self-cleaning nozzle assembly of claim 14, wherein the nano photocatalytic material is an antimicrobial nano-layer.

17. The self-cleaning nozzle assembly of claim 14, wherein said nozzle is translucent.

18. The self-cleaning nozzle assembly of claim 14, wherein said nozzle further comprises a syrup tube and a diffuser housed in said chamber.

19. The self-cleaning nozzle assembly of claim 14, wherein said nozzle further comprises a UV light source.

20. The self-cleaning nozzle assembly of claim 14, wherein said nozzle further composes a reflective coating in said chamber.
